(19) United States
(12) Patent Application Publication
(10) Pub. No.: US 2017/0207824 A1
(43) Pub. Date: Jul. 20, 2017

(54) METHODS AND APPARATUS FOR WIRELESSLY TRANSFERRING POWER

(71) Applicant: QUALCOMM INCORPORATED, SAN DIEGO, CA (US)

(72) Inventors: WILLIAM HENRY VON NOVAK, III, SAN DIEGO, CA (US); MEI-LI CHI, SAN DIEGO, CA (US)

(21) Appl. No.: 15/192,390

(22) Filed: Jun. 24, 2016

Related U.S. Application Data
(60) Provisional application No. 62/278,945, filed on Jan. 14, 2016.

(51) Int. Cl. H04B 5/00; A61N 1/378; H04W 4/00

(52) U.S. Cl. CPC H04B 5/0037; H04W 4/008; A61N 1/3787; A61B 2560/0219

(57) ABSTRACT

This disclosure provides methods and apparatus for wirelessly receiving power. An aspect of the invention is an apparatus for receiving wireless power. The apparatus comprises a first power receiver circuit and a tuning circuit. The first power receiver circuit is configured to receive power from a first wireless charging field oscillating at a first frequency different from a second frequency at which a second power receiver circuit receives power from a second wireless charging field. The tuning circuit is coupled to and configured to tune the first power receiver circuit to receive the power over a first bandwidth associated with the first frequency. The first bandwidth is fully separated from a second bandwidth associated with the second frequency over which the second power receiver circuit receives power.

### CROSS REFERENCE TO RELATED APPLICATIONS

This Application claims the benefit of U.S. Provisional Application No. 62/278,945, filed Jan. 14, 2016, and entitled “METHODS AND APPARATUS FOR WIRELESSLY TRANSFERRING POWER” and assigned to the assignee hereof. The disclosure of this prior application is considered part of this application, and is hereby incorporated by reference in its entirety.

### FIELD

The present disclosure relates generally to wireless power transfer, and more specifically to methods and apparatus for wirelessly conveying power to electronic devices that may be implanted within or worn on a user body.

### BACKGROUND

In wireless power applications, wireless power charging systems may provide the ability to charge and/or power electronic devices without physical, electrical connections, thus reducing the number of components required for operation of the electronic devices and simplifying the use of the electronic device. Such wireless power charging systems may comprise a wireless power transmitter and other transmitting circuitry configured to generate a magnetic field that may be used to wirelessly transfer power to wireless power receivers. There is a need for improved methods and apparatus for receiving wireless power transmissions by receivers in close proximity with other receivers, for example receivers in medical implants or user worn medical devices.

### SUMMARY

Various implementations of methods and devices within the scope of the appended claims each have several aspects, no single one of which is solely responsible for the desirable attributes described herein. Without limiting the scope of the appended claims, some prominent features are described herein.

An aspect of the invention is an apparatus for receiving wireless power. The apparatus comprises a first power receiver circuit and a tuning circuit. The first power receiver circuit is configured to receive power via coupling to a first wireless charging field oscillating at a first frequency different from a second frequency at which a second power receiver circuit receives power from a second wireless charging field. The tuning circuit is coupled to and configured to tune the first power receiver circuit to receive the power over a first bandwidth associated with the first frequency. The first bandwidth is fully separated (e.g., non-overlapping) from a second bandwidth associated with the second frequency over which the second power receiver circuit receives power.

Another aspect of the invention is a method for receiving power wirelessly. The method comprises receiving power from a first wireless charging field, via a first power receiver circuit, the first wireless charging field oscillating at a first frequency different from a second frequency at which a second power receiver circuit receives power from the a second wireless charging field. The method further comprises tuning, via a tuning circuit, the first power receiver circuit to receive the power over a first bandwidth associated with the first frequency, wherein the first bandwidth is non-overlapping with a second bandwidth associated with the second frequency over which the second power receiver circuit receives power.

Another aspect of the invention is another apparatus for receiving wireless power. The apparatus comprises means for receiving power from a first wireless charging field and means for tuning the means for receiving power. The means for receiving power receives power via the first wireless charging field oscillating at a first frequency different from a second frequency at which a power receiver receives power from the a second wireless charging field. The means for tuning tunes the means for receiving power to receive the power over a first bandwidth associated with the first frequency, wherein the first bandwidth is non-overlapping with a second bandwidth associated with the second frequency over which the power receiver receives power.

Another aspect of the invention is a system for transferring wireless power. The system comprises a first power receiver circuit configured to receive a first power via coupling to a first wireless charging field oscillating at a first frequency, the first power receiver circuit also comprising a first tuning circuit configured to tune the first power receiver circuit to receive the first power over a first bandwidth associated with the first frequency. The system further comprises a second power receiver circuit configured to receiver a second power coupling to a second wireless charging field oscillating at a second frequency different from the first frequency, the second power receiver circuit comprising a second tuning circuit configured to tune the second power receiver circuit to receive the second power over a second bandwidth associated with the second frequency, wherein the second bandwidth is non-overlapping with the first bandwidth associated with the first frequency over which the first power receiver circuit receives power.

### BRIEF DESCRIPTION OF THE DRAWINGS

Details of one or more implementations of the subject matter described in this specification are set forth in the accompanying drawings and the description below. Other features, aspects, and advantages will become apparent from the description, the drawings, and the claims.

FIG. 1 is a functional block diagram of a wireless power transfer system, in accordance with one exemplary implementation.

FIG. 2 is a functional block diagram of a wireless power transfer system, in accordance with another exemplary implementation.

FIG. 3 is a schematic diagram of a portion of transmit circuitry or receive circuitry of FIG. 2 including a transmit or receive antenna, in accordance with exemplary implementations.

FIG. 4 is a simplified functional block diagram of a transmitter that may be used in an inductive power transfer system, in accordance with exemplary implementations of the invention.

FIG. 5 is a simplified functional block diagram of a receiver that may be used in the inductive power transfer system, in accordance with exemplary implementations of the invention.

FIG. 6 shows a view of a wireless power transfer system 600 as applied to an area of a human body.

FIG. 7 shows a plurality of transmitters and receivers, each transmitter configured to transmit at a different respective frequency to one of the receivers.

FIG. 8 is a graph showing an exemplary frequency response for the two wireless power receivers of FIG. 6.

FIG. 9 is a process flow diagram of an exemplary method 900 for receiving wireless power, in accordance with certain aspects of the present disclosure.

The various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DETAILED DESCRIPTION

The detailed description set forth below in connection with the appended drawings is intended as a description of exemplary implementations and is not intended to represent the only implementations in which the invention may be practiced. The term “exemplary” used throughout this description means “serving as an example, instance, or illustration,” and should not necessarily be construed as preferred or advantageous over other exemplary implementations. The detailed description includes specified details for the purpose of providing a thorough understanding of the exemplary implementations. In some instances, some devices are shown in block diagram form.

Wirelessly transferring power may refer to transferring any form of energy associated with electric fields, magnetic fields, electromagnetic fields, or otherwise from a transmitter to a receiver without the use of physical electrical conductors (e.g., power may be transferred through free space). The power output into a wireless field (e.g., a magnetic field) may be received, captured by, or coupled by a “receiving coil” to achieve power transfer.

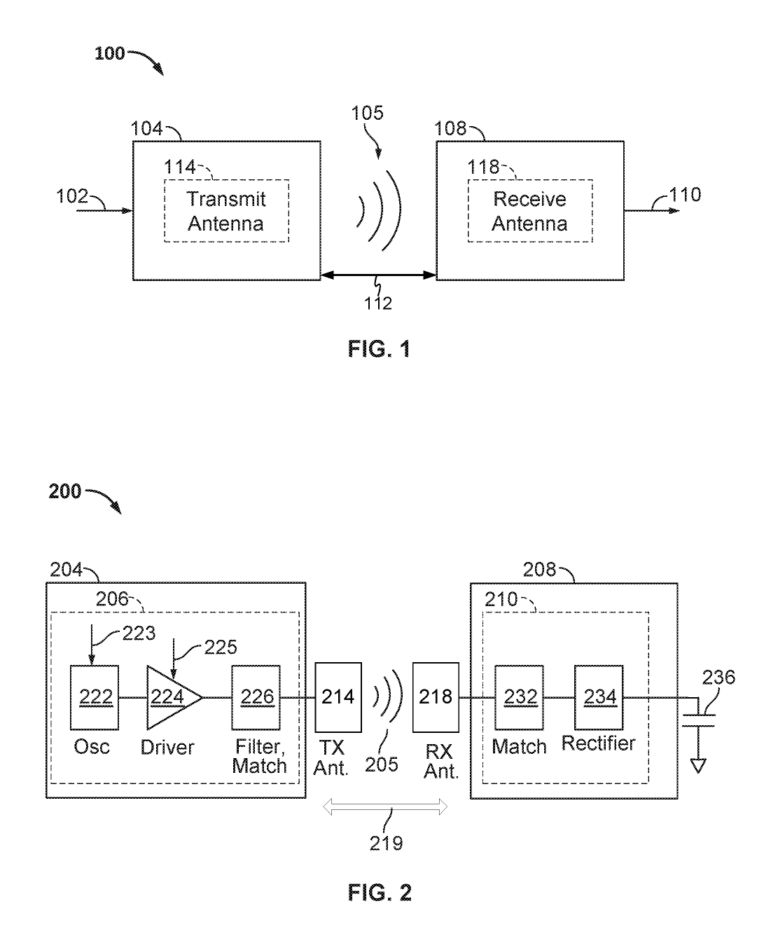

FIG. 1 is a functional block diagram of a wireless power transfer system 100, in accordance with one exemplary implementation. Input power 102 may be provided to a transmitter 104 from a power source (not shown) to generate a wireless (e.g., magnetic or electromagnetic) field 105 for performing wireless power transfer. A receiver 108 may couple to the wireless field 105 and generate output power 110 for storage or consumption by a device (not shown) coupled to the output power 110. Both the transmitter 104 and the receiver 108 are separated by a distance 112.

In one exemplary implementation, the transmitter 104 and the receiver 108 are configured according to a mutual resonant relationship. When the resonant frequency of the receiver 108 and the resonant frequency of the transmitter 104 are substantially the same or very close, transmission losses between the transmitter 104 and the receiver 108 are reduced. As such, wireless power transfer may be provided over a larger distance in contrast to purely inductive solutions that may require large antenna coils which are very close (e.g., sometimes within millimeters). Resonant inductive coupling techniques may thus allow for improved efficiency and power transfer over various distances and with a variety of inductive coil configurations.

The receiver 108 may receive power when the receiver 108 is located in the wireless field 105 produced by the transmitter 104. The wireless field 105 corresponds to a region where energy output by the transmitter 104 may be captured by the receiver 108. The wireless field 105 may correspond to the “near-field” of the transmitter 104 as will be further described below. The wireless field 105 may also operate over a longer distance than is considered “near field,” e.g. the mid-field or far field.

In the far field, power may be radiated in an electromagnetic wave having both magnetic field (H-field) and electric field (e-field) properties. Each of these fields may have dipole characteristics. A given medium may produce a fixed ration between the H-field and E-field intensities to which the medium is exposed. As distance from the transmitter generating the far field wave increases, the power decreases. Additionally, absorption of the electromagnetic wave by a receiver does not affect the transmitter (e.g., minimal or no coupling between the receiver and the transmitter). The far field may be defined as having a distance of two or more wavelengths from the transmitter. Accordingly, for a system operating at or near 1.8 GHz, the far field may correspond to a region greater than 32 cm from the transmitter.

In the near field, the H-field and the E-field of an electromagnetic field generated by the transmitter may not be related. For example, the electromagnetic field may have a strong H-field with minimal or no E-field associated with it, or vice versa. Additionally, in the near field, reception of the electromagnetic field by the receiver may affect the transmitter (e.g., the transmitter and receiver may couple to each other). This coupling, when strong, may make power transmission between the transmitter and the receiver more efficient. Furthermore, it may be difficult to focus or direct fields (e.g., form “beams” or focused magnetic or electric fields) in the near field. The near field may be defined to be within a distance of one wavelength from the transmitter. Accordingly, for a system operating at or near 1.8 GHz, the near field may correspond to a region between 0 cm and 16 cm from the transmitter.

The midfield may comprise a region between the near field and the far field that may comprise some characteristics of both the near field and the far field. For example, the midfield may provide for limited beamforming of the electromagnetic field while still allowing for efficient power transfer due to coupling between the transmitter and receiver. The midfield may be defined to be within one and two wavelengths of the transmitter, but may extend to less than one wavelength and greater than two wavelengths in some implementations. For the invention described herein, the midfield may be defined as being between 0.25 and 3 wavelengths, which may correspond to distances of 4 and 50 cm of the transmitter.

The transmitter 104 may include a transmit antenna 114 (e.g., a coil) for transmitting energy to the receiver 108. The receiver 108 may include a receive antenna or coil 118 for receiving or capturing energy transmitted from the transmitter 104. The near-field may correspond to a region in which there are strong reactance fields resulting from the currents and charges in the transmit antenna 114 that minimally radiate power away from the transmit antenna 114. The near-field may correspond to a region that is within about one wavelength (or a fraction thereof) of the transmit antenna 114.

FIG. 2 is a functional block diagram of a wireless power transfer system 200, in accordance with another exemplary implementation. The system 200 includes a transmitter 204 and a receiver 208. The transmitter 204 may include a transmit circuitry 206 that may include an oscillator 222, a driver circuit 224, and a filter and matching circuit 226. The oscillator 222 may be configured to generate a signal at a desired frequency that may be adjusted in response to a frequency control signal 223. The oscillator 222 may provide the oscillator signal to the driver circuit 224. The driver circuit 224 may be configured to drive the transmit antenna 214 at, for example, a resonant frequency of the transmit antenna 214 based on an input voltage signal (V_{D}) 225. The driver circuit 224 may be a switching amplifier configured to receive a square wave from the oscillator 222 and output a sine wave (e.g., a wave with sinusoidal oscillations). For example, the driver circuit 224 may be a class E amplifier.

The filter and matching circuit 226 may filter out harmonics or other unwanted frequencies and match the impedance of the transmitter 204 to the impedance of the transmit antenna 214. As a result of driving the transmit antenna 214, the transmit antenna 214 may generate a wireless field 205 to wirelessly output power at a level sufficient for charging a battery 236.

The receiver 208 may include a receive circuitry 210 that may include a matching circuit 232 and a rectifier circuit 234. The matching circuit 232 may match the impedance of the receive circuitry 210 to the receive antenna 218. The rectifier circuit 234 may generate a direct current (DC) power output from an alternate current (AC) power input to charge the battery 236, as shown in FIG. 2. The receiver 208 and the transmitter 204 may additionally communicate on a separate communication channel 219 (e.g., Bluetooth, ZigBee, cellular, etc.). The receiver 208 and the transmitter 204 may alternatively communicate via in-band signaling using characteristics of the wireless field 205.

The receiver 208 may be configured to determine whether an amount of power transmitted by the transmitter 204 and received by the receiver 208 is appropriate for charging the battery 236.

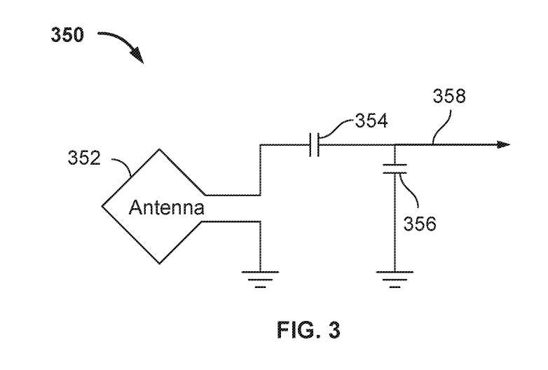

FIG. 3 is a schematic diagram of a portion of the transmit circuitry 206 or the receive circuitry 210 of FIG. 2 including a transmit or receive antenna, in accordance with exemplary implementations. As illustrated in FIG. 3, a transmit or receive circuitry 350 may include an antenna 352. The antenna 352 may also be referred to or be configured as a “loop” antenna 352. The antenna 352 may also be referred to herein or be configured as a “magnetic” antenna or an induction coil. The term “antenna” generally refers to a component that may wirelessly output or receive energy for coupling to another “antenna.” The antenna may also be referred to as a coil of a type that is configured to wirelessly output or receive power. As used herein, the antenna 352 is an example of a “power transfer component” of a type that is configured to wirelessly output and/or receive power.

The antenna 352 may include an air core or a physical core such as a ferrite core (not shown).

The transmit or receive circuitry 350 may form/include a resonant circuit. The resonant frequency of the loop or magnetic antennas is based on the inductance and capacitance. Inductance may be simply the inductance created by the antenna 352, whereas, capacitance (e.g., a capacitor) may be electrically connected to antenna 352 create a resonant structure configured to resonate at a desired resonant frequency (or further inductors may additional be added). As a non-limiting example, a capacitor 354 and a capacitor 356 may be added to the transmit or receive circuitry 350 to create a resonant circuit. For a transmit circuitry, a signal 358 may be an input at a resonant frequency to cause the antenna 352 to generate a wireless field 105/205. For receive circuitry, the signal 358 may be an output to power or charge a load (not shown). For example, the load may comprise a wireless device configured to be charged by power received from the wireless field.

Other resonant circuits formed using other components are also possible. As another non-limiting example, a capacitor may be placed in parallel between the two terminals of the circuitry 350.

Referring to FIGS. 1 and 2, the transmitter 104/204 may output a time varying magnetic (or electromagnetic) field with a frequency corresponding to the resonant frequency of the transmit antenna 114/214. When the receiver 108/208 is within the wireless field 105/205, the time varying magnetic (or electromagnetic) field may induce a current in the receive antenna 118/218. As described above, if the receive antenna 118/218 is configured to resonate at the frequency of the transmit antenna 114/214, energy may be efficiently transferred. The AC signal induced in the receive antenna 118/218 may be rectified as described above to produce a DC signal that may be provided to charge or to power a load.

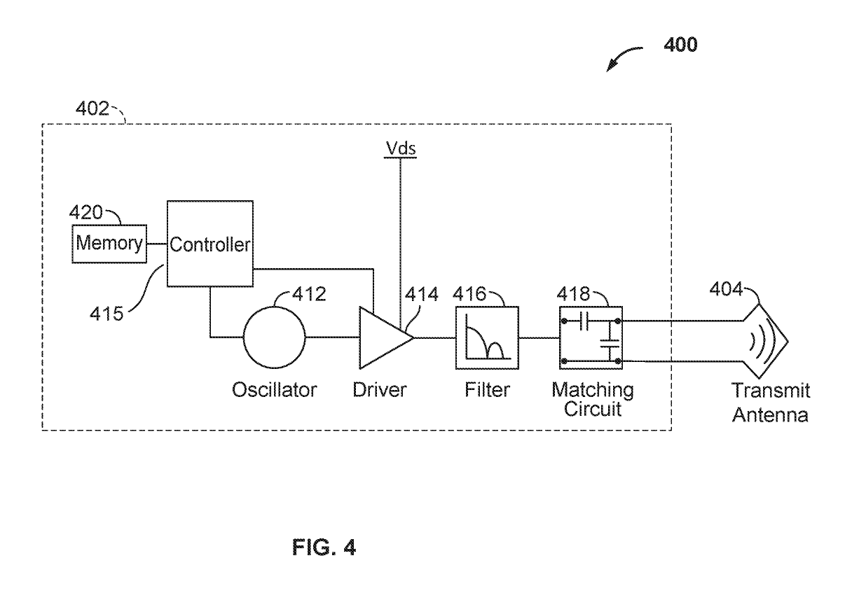

FIG. 4 is a simplified functional block diagram of a transmitter that may be used in an inductive power transfer system, in accordance with exemplary implementations of the invention. As shown in FIG. 4, the transmitter 400 includes transmit circuitry 402 and a transmit antenna 404 operably coupled to the transmit circuitry 402. The transmit antenna 404 may be configured as the transmit antenna 214 as described above in reference to FIG. 2. In some implementations, the transmit antenna 404 may be a coil (e.g., an induction coil). In some implementations, the transmit antenna 404 may be associated with a larger structure, such as a table, mat, lamp, or other stationary configuration. The transmit antenna 404 may be configured to generate an electromagnetic or magnetic field. In an exemplary implementation, the transmit antenna 404 may be configured to transmit power to a receiver device within a charging region at a power level sufficient to charge or power the receiver device.

The transmit circuitry 402 may receive power through a number of power sources (not shown). The transmit circuitry 402 may include various components configured to drive the transmit antenna 404. In some exemplary implementations, the transmit circuitry 402 may be configured to adjust the transmission of wireless power based on the presence and constitution of the receiver devices as described herein. As such, the transmitter 400 may provide wireless power efficiently and safely.

The transmit circuitry 402 may further include a controller 415. In some implementations, the controller 415 may be a micro-controller. In other implementations, the controller 415 may be implemented as an application-specified integrated circuit (ASIC). The controller 415 may be operably connected, directly or indirectly, to each component of the transmit circuitry 402. The controller 415 may be further configured to receive information from each of the components of the transmit circuitry 402 and perform calculations based on the received information. The controller 415 may be configured to generate control signals for each of the components that may adjust the operation of that component. As such, the controller 415 may be configured to adjust the power transfer based on a result of the calculations performed by it.

The transmit circuitry 402 may further include a memory 420 operably connected to the controller 415. The memory 420 may comprise random-access memory (RAM), electrically erasable programmable read only memory (EEPROM), flash memory, or non-volatile RAM. The memory 420 may be configured to temporarily or permanently store data for use in read and write operations performed by the controller 415. For example, the memory 420 may be configured to store data generated as a result of the calculations of the controller 415. As such, the memory 420 allows the controller 415 to adjust the transmit circuitry 402 based on changes in the data over time.

The transmit circuitry 402 may further include an oscillator 412 operably connected to the controller 415. The oscillator 412 may be configured as the oscillator 222 as described above in reference to FIG. 2. The oscillator 412 may be configured to generate an oscillating signal (e.g., radio frequency (RF) signal) at the operating frequency of the wireless power transfer. In some exemplary implementations, the oscillator 412 may be configured to operate at the 6.78 MHz ISM frequency band. The controller 415 may be configured to selectively enable the oscillator 412 during a transmit phase (or duty cycle). The controller 415 may be further configured to adjust the frequency or a phase of the oscillator 412 which may reduce out-of-band emissions, especially when transitioning from one frequency to another. As described above, the transmit circuitry 402 may be configured to provide an amount of power to the transmit antenna 404, which may generate energy (e.g., magnetic flux) about the transmit antenna 404.

The transmit circuitry 402 may further include a driver circuit 414 operably connected to the controller 415 and the oscillator 412. The driver circuit 414 may be configured as the driver circuit 224 as described above in reference to FIG. 2. The driver circuit 414 may be configured to drive the signals received from the oscillator 412, as described above.

The transmit circuitry 402 may further include a low pass filter (LPF) 416 operably connected to the transmit antenna 404. The low pass filter 416 may be configured as the filter portion of the filter and matching circuit 226 as described above in reference to FIG. 2. In some exemplary implementations, the low pass filter 416 may be configured to receive and filter an analog signal of current and an analog signal of voltage generated by the driver circuit 414. The analog signal of current may comprise a time-varying current signal, while the analog signal of current may comprise a time-varying voltage signal. In some implementations, the low pass filter 416 may alter a phase of the analog signals. The low pass filter 416 may cause the same amount of phase change for both the current and the voltage, canceling out the changes. In some implementations, the controller 415 may be configured to compensate for the phase change caused by the low pass filter 416. The low pass filter 416 may be configured to reduce harmonic emissions to levels that may prevent self-jamming. Other exemplary implementations may include different filter topologies, such as notch filters that attenuate specified frequencies while passing others.

The transmit circuitry 402 may further include a fixed impedance matching circuit 418 operably connected to the low pass filter 416 and the transmit antenna 404. The matching circuit 418 may be configured as the matching portion of the filter and matching circuit 226 as described above in reference to FIG. 2. The matching circuit 418 may be configured to match the impedance of the transmit circuitry 402 (e.g., 50 ohms) to the transmit antenna 404. Other exemplary implementations may include an adaptive impedance match that may be varied based on measurable transmit metrics, such as the measured output power to the transmit antenna 404 or a DC current of the driver circuit 414. The transmit circuitry 402 may further comprise discrete devices, discrete circuits, and/or an integrated assembly of components.

Transmit antenna 404 may be implemented as an antenna strip with the thickness, width and metal type selected to keep resistance losses low.

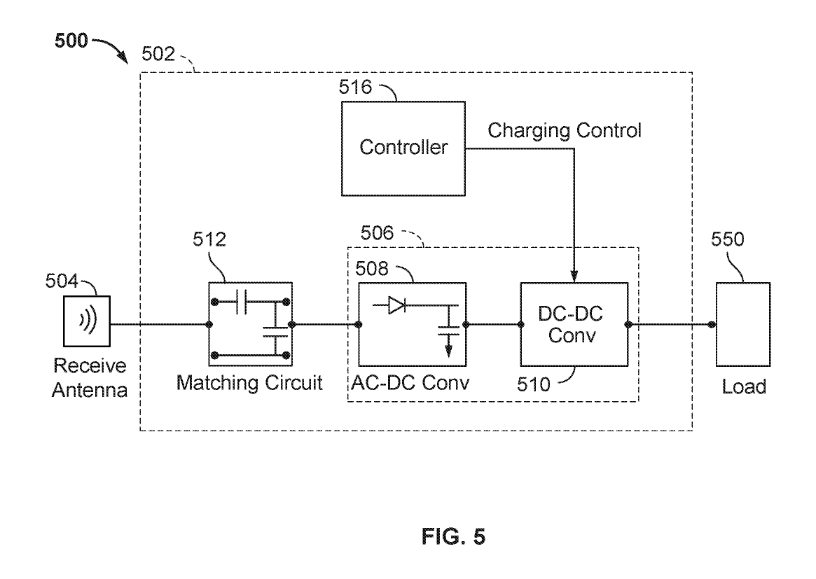

FIG. 5 is a block diagram of a receiver, in accordance with an implementation of the present invention. As shown in FIG. 5, a receiver 500 includes a receive circuitry 502, a receive antenna 504, and a load 550. The receiver 500 further couples to the load 550 for providing received power thereto. Receiver 500 is illustrated as being external to device acting as the load 550 but may be integrated into load 550. The receive antenna 504 may be operably connected to the receive circuitry 502. The receive antenna 504 may be configured as the receive antenna 218 as described above in reference to FIG. 2. In some implementations, the receive antenna 504 may be tuned to resonate at a frequency similar to a resonant frequency of the transmit antenna 404, or within a specified range of frequencies, as described herein. The receive antenna 504 may be similarly dimensioned with transmit antenna 404 or may be differently sized based upon the dimensions of the load 550. The receive antenna 504 may be configured to couple to the magnetic field generated by the transmit antenna 404, as described above, and provide an amount of received energy to the receive circuitry 502 to power or charge the load 550.

The receive circuitry 502 may be operably coupled to the receive antenna 504 and the load 550. The receive circuitry may be configured as the receive circuitry 210 as described above in reference to FIG. 2. The receive circuitry 502 may be configured to match an impedance of the receive antenna 504, which may provide efficient reception of wireless power. The receive circuitry 502 may be configured to generate power based on the energy received from the receive antenna 504. The receive circuitry 502 may be configured to provide the generated power to the load 550. In some implementations, the receiver 500 may be configured to transmit a signal to the transmitter 400 indicating an amount of power received from the transmitter 400.

The receive circuitry 502 may include a processor-signaling controller 516 configured to coordinate the processes of the receiver 500 described below.

The receive circuitry 502 provides an impedance match to the receive antenna 504. The receive circuitry 502 includes power conversion circuitry 506 for converting a received energy into charging power for use by the load 550. The power conversion circuitry 506 includes an AC-to-DC converter 508 coupled to a DC-to-DC converter 510. The AC-to-DC converter 508 rectifies the AC energy signal received at the receive antenna 504 into a non-alternating power while the DC-to-DC converter 510 converts the rectified AC energy signal into an energy potential (e.g., voltage) that is compatible with the load 550. Various AC-to-DC converters are contemplated including partial and full rectifiers, regulators, bridges, doublers, as well as linear and switching converters.

The receive circuitry 502 may further include a matching circuit 512. The matching circuit 512 may comprise one or more resonant capacitors in either a shunt or a series configuration. In some implementations these resonant capacitors may tune the receive antenna to a specific frequency or to a specific frequency range (e.g., a resonant frequency). In some implementations, the resonant capacitors (and other resonant components) may not explicitly faint the matching circuit 512. In some implementations, a series tuned receiver may maintain a fixed voltage given exposure to a fixed field. This fixed voltage may simplify design of high power receivers, as power electronics prefer minimal variation in operating voltages. In some implementations, a shunt tuned receiver may increase the voltage received for use within the receiver. In a low power, low field receiver, the shunt tuning can boost an unusable 10 mV received power signal into a useful 2 volt signal. Some receivers may comprise a combination of series tuned and shunt tuned circuits or components.

The load 550 may be operably connected to the receive circuitry 502. The load 550 may be configured as the battery 236 as described above in reference to FIG. 2. In some implementations the load 550 may be external to the receive circuitry 502. In other implementations the load 550 may be integrated into the receive circuitry 502.

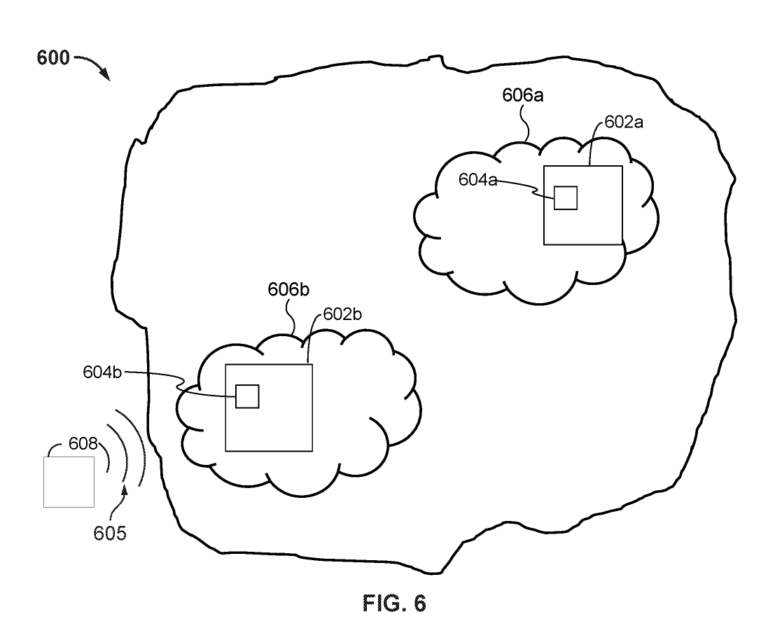

FIG. 6 shows a view of a wireless power transfer system 600 as applied to an area of a human body. The system 600 comprises two implants 602a and 602b that each comprises a wireless power receiver 604a and 604b, respectively. The receivers 604a and 604b each receive power wirelessly from a transmitter 608 via a wireless field 605. For example, the transmitter 608 may be configured to generate an alternating magnetic field for transferring power in the near-field. The receivers 604a and 604b each correspond to the receiver 500 of FIG. 5. The transmitter 608 corresponds to the transmitter 400 of FIG. 4. The area comprises two regions 606a and 606b. The two regions 606a and 606b each correspond to a different type of bodily fluid or tissue within the area of the body. For example, region 606a may correspond to muscle tissue while the region 606b may correspond to bone. The implant 602a with the receiver 604a is located within the region 606a, while the implant 602b with the receiver 604b is located within the region 606b.

The area of the body of the system 600 may be replaced by an area of any other living body within which one or more functions may be desired to be monitored or controlled. In the area of the human body as depicted in FIG. 6, the implants 602a and 602 (e.g., comprising various electronic devices) may be implanted or worn to control or monitor various functions, signals, or conditions of the body. Each implant 602 may comprise the receiver 604. The electronic devices of the implant 602 may correspond to the load 550 of the receiver 604.

The implants 602 may allow for the diagnosis and/or treatment of diseases and/or various other conditions. In some implementations, the implants 602 may be used for medical “neuromodulation,” where the implants 602 attach to nerves of the body and monitor or stimulate the nerves to which they are attached. In some implementations, the implants 602 may control or regulate a status or a chemical value (e.g., control an introduction of a chemical) of the body. For example, the implants 602 may monitor a brain or nervous system and deliver electrical stimulation or medication to relieve pain and/or restore functions. Alternatively, or additionally, the implants 602 may comprise insulin monitors, insulin injectors, hearing aids, or pacemakers, among other implanted or wearable devices.

In some implementations, the implants 602 may utilize primary batteries as a power source. However, as the batteries require replacement, replacement of the batteries in the implants 602 may require surgery to perform the replacement. Accordingly, alternate, or additional, methods of powering the implants 602 are desired. Wireless charging and/or power transfer may provide a safer and less invasive method of powering such implants 602 in the long term. The transmitter 608 may transfer power wirelessly via the wireless field 605 to charge or power the receivers 604a and 604b of such implants 602.

However, wirelessly charging or powering the implants 602 may cause problems when multiple implants 602 are in close proximity with each other. For example, the different implants 602a and 602b may each be designed having different charge or power parameters. Additionally, positions of the first and second implants 602a and 602b may be determined by the anatomy of the body and may not be alterable to ease charging or powering requirements. Thus, the two implants 602a and 602b may be in close proximity with each other when one or both of them needs to be charged or powered. Such close proximity may result in the two implants 602a and 602b not being able to be independently charged without receiving interference while the other implant 602a or 602b is being charged. Exposing both the implants 602a and 602b to a single wireless field may damage one or both of the implants 602a and 602b. Charging and powering the implants 602 will be described below as charging or powering the respective receivers 604 of the implants 602.

For example, the wireless field 605 that charges or powers the first receiver 604a may have a first set of parameters. The second receiver 604b may have a second set of parameters different from the first set of parameters. When the first receiver 604a is in close proximity with the second receiver 604b, the wireless field 605 may expose the second receiver 604b to conditions, e.g., field strengths, etc., that may damage the second receiver 604b because they are outside the second set of parameters. In an implementation, the first receiver 604a couples with the wireless field 605 to wirelessly receive power at a first power level. However, the second receiver 604b, also within range of the wireless field 605, may inadvertently couple with the wireless field 605 at a second, higher power level that may damage the second receiver 604b. This damage may result in improper operation of the implants 602.

The methods and systems defined herein charge and power each of the implants 602 positioned in close proximity to each other without a risk of damage to neighboring implants 602. The transmitter 608 may generate the wireless field 605 at a given frequency or at multiple frequencies within a spectrum of frequencies. The spectrum may include a range of frequency values (e.g., 1.0 GHz to 2.4 GHz) available for use to transfer power within the area of the body. The wireless field 605 may transfer power, via the receivers 604a and 604b, to the implants 602a and 602b, respectively, within the area of the body exposed to the wireless field 605. In some implementations, the transmitter 608 generates the wireless field 605 at a frequency of 1.2 GHz to charge any receivers 604 tuned to have a resonant frequency of 1.2 GHz.

In some implementations, the receiver 604a receives power via the wireless field 605 at a first tuned resonant frequency. The receiver 604b receives power via the wireless field 605 at a second tuned resonant frequency different from the first tuned resonant frequency. The frequency spectrum described above may include the two tuned resonant frequencies of the receivers 604a and 604b, respectively. In some implementations, the frequency spectrum is divided according to a number of tuned receivers 604 within the area of the wireless field 605. For example, when there are nine tuned receivers 604 within the area of the wireless field 605, the spectrum of frequencies may be divided into nine unique resonant frequencies. Each of the nine receivers 604 may be tuned to only one of the nine unique resonant frequencies. The receivers 604 are tuned to determine the resonant frequencies. In some implementations, the receivers 604 may be adjustable to allow for adjustment of the respective resonant frequencies and Q factors. In some implementations, the respective processors of the receivers 604 may adjust the receivers based on a quantity of the other receiver 604 within an area of the magnetic field 605. Accordingly, respective resonant frequencies and Q factors of the receivers 604 may be adjusted so as to allow for additional receivers 604 within the spectrum of frequencies. In some implementations, the receivers 604 may each receive communications from other receivers 604 or the transmitter 608 to determine the quantity and/or operating frequency of other receivers 604 within the area of the magnetic field 605. In some implementations, the receivers 604 may be independently configured to determine the quantity and/or operating frequency of other receivers 604 within the area of the magnetic field 605. In some implementations, the receivers 604 may be configured to identify a quantity of other wireless power fields (e.g., magnetic fields) that are generated in an area of the body within which the receiver 604 is implanted. Accordingly, the receiver 604 may adjust its bandwidth to compensate for other fields generated in its vicinity so it may protect itself from damage. In some implementations, the receivers 604 may communicate with each other to instruct other receivers 604 to adjust their bandwidth (e.g., via their matching circuits) to minimize damage. For example, when a receiver 604c (not shown) is added to the system 600, the receiver 604c may send a message to one or both of the receivers 604a and 604b instructing them to adjust their bandwidths in view of the operating frequency of the receiver 604c.

The receiver 604 may be tuned to accept or respond to its resonant frequency while significantly discriminating or attenuating other frequencies. A quality (“Q”) factor of the receivers 604 may be a measure how well the receiver 604 responds to its resonant frequency while attenuating other frequencies. For example, a Q factor of less than 20 may be characteristic of a receiver 604 (e.g., a broadband power receiver) with poor selectivity (e.g., poor abilities to discriminate or attenuate other frequencies besides its resonant frequency). The Q factor may be reduced by a poor quality of the components or high resistance of the receive antenna.

The Q factor of the receiver 604, described above, may impact a frequency bandwidth within which the receiver 604 efficiently and effectively receives the generated wireless field 605. A low Q factor indicates that the receiver 604 has a broader bandwidth, and is less discriminating of frequencies other than its resonant frequency. A high Q factor indicates that the receiver 604 has a narrower bandwidth and is more discriminating of frequencies other than its resonant frequency. A narrow bandwidth that causes significant discrimination of frequencies other than the resonant frequency is desirable. The narrow bandwidth allows the receiver 604 to ignore (e.g., discriminate or attenuate) the wireless field 605 that is conveying power at a frequency outside the bandwidth of the resonant frequency of the receiver 604. The discrimination may prevent damage that may otherwise result from exposure of the receiver 604 to wireless fields 605 of other frequencies. By implementing the narrow bandwidth, the receiver 604 may effectively isolate its response to only wireless fields 605 operating at its resonant frequency and not be damaged or significantly affected by any wireless fields 605 operating at neighboring frequencies.

In an example of an RLC circuit of the receiver 604a, the R (resistance)=10 ohms (for example, representing parasitic losses from components of the receiver 604a), the L (inductance)=0.493 μH (for example, representing a 6 turn, 8 mm diameter coil), and the C (capacitance)=0.018 pF. Applying these values to Equations 1-1 through 1-3 below produces a resonant frequency of 1.7 GHz and a Q factor of 84 for the receiver 604a. The Q factor of 84 would allow for a 50 MHz channel spacing between resonant frequencies of the receiver 604 with an attenuation of 1000 to 1 (i.e. a 100 volt signal would be attenuated to 0.1 volts).

$$\begin{matrix}{ω}_{0} = \frac{1}{\sqrt{LC}} & Equation 1 \text{-} 1 \\ Q = \frac{{ω}_{0} L}{R} & Equation 1 \text{-} 2 \\ B = \frac{R}{L} & Equation 1 \text{-} 3\end{matrix}$$

Where ω₀=resonant frequency (F) of the receiver;

L=inductance of the receiver;

C=capacitance of the receiver;

R=resistance of the receiver; and

B=bandwidth of the receiver.

Varying the Q factors of each of the receivers 604a and 604b (e.g., changing one or more of the R, L, or C values of the receiver 604) may control the respective resonant frequency of the receivers 604a and 604b within a broader or narrower bandwidth range. Thus the different receivers 604 may each have different R, L, or C values and may each have different resonant frequencies and Q factors. In the example of the receiver 604a having the resonant frequency of 1.7 GHz and a Q factor of 84, the bandwidth to which the receiver responds may be less than 50 MHz (e.g., 20 MHz). Assume that the receiver 604b shares the resonant frequency, Q factor, and bandwidth with receiver 604a but has different charging or powering requirements. If the receiver 604b is exposed to the wireless field 605 that charges the receiver 604a at 1.7 GHz, that exposure may cause an overvoltage in the receiver 604b, for example, an overvoltage of 100V. The 100V overvoltage would likely damage the receiver 604b. However, tuning the receiver 604b to a unique resonant frequency, for example 1.65 GHz with a Q factor of 84 and a bandwidth of 20 MHz, the 100V overvoltage may be attenuated. The voltage attenuation of the receiver 604b at the 1.65 GHz frequency is 40 dB greater than the voltage attenuation at 1.7 GHz, corresponding to a factor of 1000. Accordingly, the receiver 604b having the resonant frequency at 1.65 GHz would reduce a potential overvoltage by the factor of 1000. Thus, the 100V overvoltage would be reduced to a reasonable 0.1V value that the receiver 604b can handle. Accordingly, the receiver 604b with a unique resonant frequency and narrow bandwidth is not damaged by the overvoltage condition that may otherwise damage the receiver 604a at a shared resonant frequency.

In some implementations, the transmitter 608 may generate the wireless field 605 to charge one receiver 604 at a single frequency. Alternatively, or additionally, the transmitter 608 may simultaneously generate the wireless field 605 to charge each receiver 604 of a plurality of receivers 604 at one of a plurality of frequencies. This multiple frequency charging may utilize frequency division multiplexing (“FDM”) to accommodate simultaneously charging or powering multiple devices each at individual frequencies. FDM may allow multiple receivers 604 to charge or operate simultaneously without providing too much power/voltage to any one receiver 604. In FDM, the transmitter 608 may generate the wireless field 605 at multiple frequencies that correspond to the resonant frequency of each receiver 604 that is charged by the FDM transmitter 608. Accordingly, each receiver 604 can have its power/voltage level tailored to its needs. The narrow bandwidths of each receiver 604 minimize the likelihood that the receiver 604 will be exposed to a wireless field 605 that may generate an overvoltage in the receiver 604.

In some implementations, the transmitter 608 may be configured to identify or select one or more frequencies at which it will generate the wireless field 605. In some implementations, a controller and/or a memory of the transmitter 608 may perform the identification or selection. In some implementations, the frequencies may be identified or selected based on one or more of the following factors:
Water-absorption frequency: water absorbs signals at 2.4 GHz; accordingly, 2.4 GHz may act as an upper or lower limit
Regulatory limitations: there will almost certainly be unusable frequencies dictated by local regulatory requirements.
The Q factor of the receivers 604a and 604b: the Q factors of the resonators in the receivers 604a and 604b may determine how far apart the frequencies generated by the transmitter 608 must be spaced.
A resonant frequency of the receivers 604: the receivers 604 may be configured to receive wireless power from only the wireless field 605 at a given frequency.
Type of bodily fluid or tissue to be penetrated: See Table 1 below for examples of optimal frequencies for various selected bodily fluids or tissues.

TABLE 1

| Tissue type | Optimal frequency (GHz) |
|---|---|
| Blood | 1.58 |
| Bone (cancellous) | 1.70 |
| Fat (infiltrated) | 2.68 |
| Fat (not infiltrated) | 3.86 |
| Heart | 1.68 |
| Liver | 1.70 |
| Lung (deflated) | 1.72 |
| Muscle | 1.76 |
| Skin (dry) | 1.98 |
| Skin (wet) | 1.79 |

Tissue may comprise any type of tissue within a human body (e.g., epithelial, connective, muscular, and nervous tissue or muscle tissue, bone tissue, organ tissue, blood tissue, or fat tissue).

In some implementations, the transmitter 608 may receive a communication or feedback signal from each receiver 604 within range of its wireless field 605. The communication or feedback signal may include acceptable power levels for each receiver 604. In some implementations, the processor of the transmitter may set the transmission parameters independently for each receiver 604 based on the feedback or communications. In some implementations, the communication or feedback signal may comprise an out-of-band link.

Given the factors listed above, there may be a range of frequencies that are acceptable for use. Table 2 below provides an example of the frequencies assigned to a number of receivers 604a-604c, assuming 100 MHz separation. The 100 MHz separate assumes each receiver 604 has a bandwidth of less than 100 MHz, outside of which the wireless field 605 is ignored or non-effective. Accordingly, in some implementations, three individual transmitters 608 may each generate wireless fields 605 at one of these frequencies to charge or power the receivers 604 having these respective resonant frequencies. Alternatively, or additionally, an FDM transmitter 608 may simultaneously generate wireless fields 605 at each of these frequencies to charge or power the receivers 604 with these respective resonant frequencies.

TABLE 2

| Implant number | Frequency | Notes |
|---|---|---|
| 604a | 1.6 GHz | Better for blood implants |
| 604b | 1.7 GHz | Better for chest implants |
| | | (bone/muscle/lung) |
| 604c | 1.8 GHz | Better for subcutaneous |
| (not shown) | | implants (skin) |

As discussed herein, the receivers 604a and 604b of implants 602a and 602b, respectively, may be tuned and/or otherwise configured to operate at specific frequencies. For example, each of the receivers 604a and 604b may comprise matching circuits (not shown) as described in reference to FIG. 5. However, the matching circuits of the receivers 604a and 604b may be tuned to different frequencies within the spectrum of frequencies described herein. For example, the receiver 604a, implanted in muscle tissue, may comprise its matching circuit including components in a shunt configurations resulting in a tuned frequency of approximately 1.70 GHz. The receiver 604b, implanted in bone, may comprise its matching circuit that includes components in a shunt or series configuration resulting in tuned frequency of approximately 1.76 GHz. In some implementations, each of the receivers 604a and 604b may be configured to operate with a bandwidth 50 MHz, such that both receivers 604a and 604b are configured to ignore the frequency of the other receiver 604a and 604b (e.g., 1.7 GHz+/−50 MHz does not overlap with the 1.76 GHz). Thus, the receivers 604a and 604b are isolated from each other.

In some implementations, one or both of the receivers 604a and 604b may monitor and/or adjust their bandwidths in relation to a quantity of other power receiver circuits, tuned frequencies of the other or available frequencies within the spectrum. For example, the receiver 604a may determine that there are additional receivers 604 in its vicinity as compared to when the receiver 604a was originally or previously implanted and/or configured. Based on the determination of additional receivers 604 in the vicinity, the receiver 604a may determine whether or not it needs to adjust its bandwidth to further or better isolate itself from the additional receivers 604. For example, the receiver 604a operating at 1.70 GHz with a bandwidth of 50 MHz may detect one of the additional receivers 604 configured with an operating frequency of 1.68 GHz (e.g., implanted in the heart, per Table 1). Based on this detection, the receiver 604a may adjust its bandwidth to be less than 20 MHz (e.g., 1.70 GHz-1.68 GHz=20 MHz) to isolate itself from the one additional receiver 604 operating at 1.68 GHz.

In some implementations, the receiver 604a may be configured to adjust its bandwidth by changing a configuration of its matching circuit. For example, the receiver 604a may change between a shunt configuration and a series configuration of components within its matching circuit. This may cause components to be reconfigured to result in a different Q factor for the receiver, which may change the bandwidth of the receiver. In some implementations, the receiver 604a may select different components (e.g., capacitors, etc.) in either the shunt or series configurations based on the desired operating frequency and/or desired bandwidth.

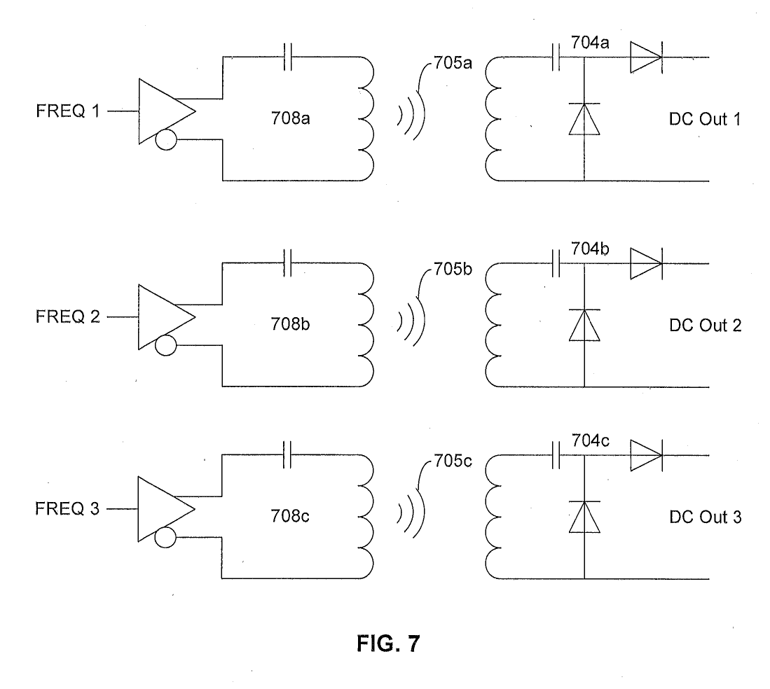

FIG. 7 shows a system 700 comprising a plurality of transmitters 708a-708c and receivers 704a-704c. Each transmitter 708 of the plurality of transmitters 708a-708c generates a wireless field 705 at a different frequency to power or charge one receiver 704 of the plurality of receivers 704a-704c. Each of the transmitters 708a-708c corresponds to the transmitter 608 of FIG. 6. Each of the receivers 704a-704c corresponds to the receivers 604a and 604b of FIG. 6.

Each of the transmitters 708a-708c generates the wireless field 705a-705c, respectively, at one of the frequencies shown above in Table 2. For example, the transmitter 708a may be tuned to generate the wireless field 705a at 1.6 GHz, the transmitter 708b may be tuned to generate the wireless field 705b at 1.7 GHz, and the transmitter 708c may be tuned to generate the wireless field 705c at 1.8 GHz. Additionally, each of the receivers 704a-704c may receive wireless power from the wireless fields 705a-705c, respectively, at a tuned resonant frequency corresponding to one of the frequencies of Table 2. For example, the receiver 704a may be tuned to have a resonant frequency at 1.6 GHz, while the receivers 704b and 704c may be tuned to have resonant frequencies at 1.7 GHz and 1.8 GHz, respectively. Additionally, each of the receivers 704a-704c may have Q factors that result in bandwidths of less than 100 MHz. Thus, as described above, each of the receivers 704a-704c largely ignore or are not damaged by wireless fields at frequencies other than their tuned resonant frequencies. For example, in conjunction with the example provided in relation to FIG. 6 above, the receivers 704a-704c may be configured to reduce potential overvoltage signals from wireless fields at “adjacent” frequencies to manageable levels that do not result in damage to the receivers 704a-704c. In some implementations, the receivers 704a-704c may comprise different shunt or series configurations (though not shown in this figure) that may result in different Q factors and may result in different bandwidths based on the specific configuration.

As discussed above, in some implementations, the transmitters 708a-708c may be replaced by a single transmitter (not shown in FIG. 7) that is capable of frequency division multiplexing (FDM). Accordingly, the single transmitter may simultaneously charge each of the receivers 704a-704c at their respective frequencies and power levels without risk of damaging one or more of the receivers 704a-704c.

Thus, the use of receivers 704a-704c that restrict or exclude frequencies may allow the receivers 704a-704c to be in proximity with other receivers without risk of damage. As the receivers 704a-704c may only interact with wireless fields 705 at the resonant frequencies of the receivers 704a-704c, the receivers 704a-704c may ignore the damaging wireless fields 705 and thus be safely exposed to such wireless fields 705. Accordingly, the receivers 704a-704c may be designed having sufficiently high Q factors to isolate or restrict themselves to sufficiently narrow bandwidths to be safe from frequencies outside their designed bandwidth. Such an implementation of controlled wireless charging or powering is simpler and cheaper than using phased-array or directional antennas for similar purposes of safe wireless power transmission to receivers in close proximity.

The methods and systems described herein can expand to cover to frequency spectrums of varying sizes. For example, with 100 MHz spacing (which requires a relatively low Q factor for the receivers) and lower and upper frequency limits of 1.5 and 2.4 GHz, respectively, nine different frequencies can be utilized. Accordingly, nine different devices with receivers 704 each having bandwidths of 100 MHz or less may be implanted or worn in a region exposed to the wireless field 705. Increasing the Q factor of the receiver 704 resonator circuit through use of high quality capacitors and/or widely spaced turns in the receiver coil can further reduce the bandwidth of the receiver 704. A reduced bandwidth may allow the frequencies to be packed more tightly, increasing a number of available frequencies for use within the same spectrum. The methods and systems described herein will work with either series or shunt tuned receivers.

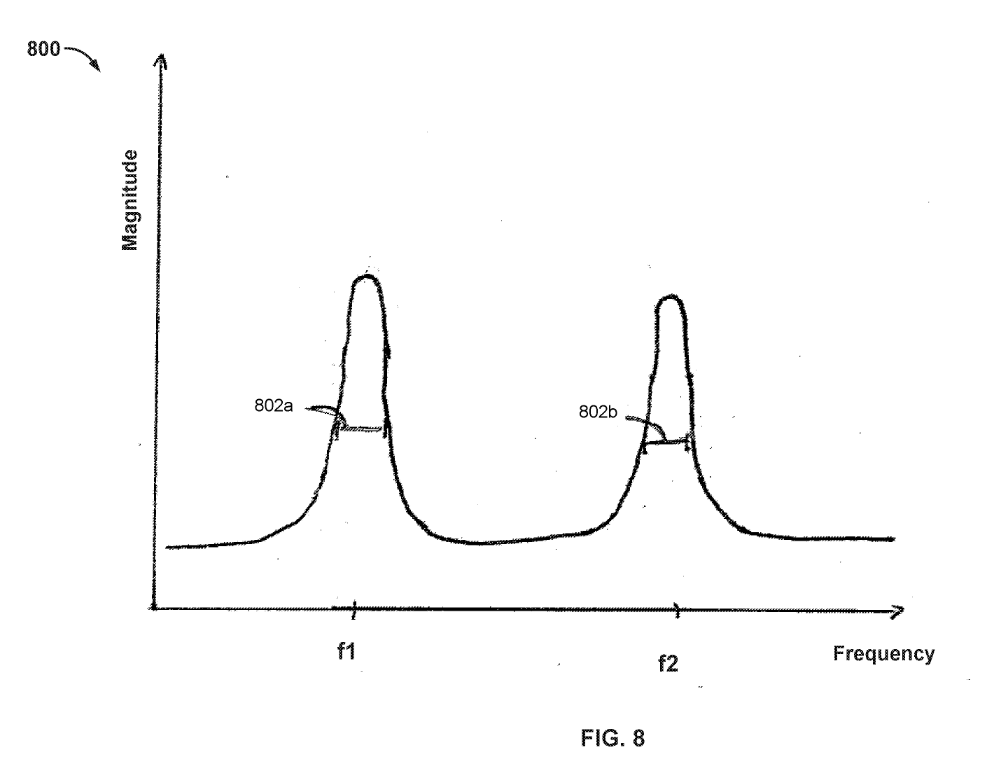

FIG. 8 is a graph 800 showing an exemplary frequency response for the two wireless power receivers 604a and 604b of FIG. 6, in accordance with an exemplary implementation. The graph shows frequency of a wireless field (e.g., wireless field 605) generated by a transmitter (e.g., transmitter 608) along the x-axis and magnitude along the y-axis. The frequency f1 may correspond to the resonant frequency of the receiver 604a, while the frequency f2 may correspond to the resonant frequency of the receiver 604b. Bandwidths 802a and 802b may correspond to the effective bandwidths of the receivers 604a and 604b, respectively. As shown, the bandwidths 802a and 802b of the respective receivers 604a and 604b may be of similar, narrow widths. In some implementations, the receivers 604a and 604b may have different respective bandwidths. The narrow bandwidths 802a and 802b allow the respective receivers 604a and 604b to effectively ignore power received at the frequency of the other receiver 604a or 604b. Accordingly, exposure of the receiver 604a to a strong wireless field at 12 (or vice versa) may not result in an overvoltage of the receiver 604a. There is no overvoltage at the receiver 604a because the receiver 604a is tuned to have a resonant frequency that reduces the overvoltage by a specified factor. For example, if f1=1.65 GHz and f2=1.7 GHz, that factor may be 1000, as described above in the example described in reference to FIG. 6. More particularly, the bandwidth 802a of the receiver 604a is narrow enough that it does not overlap with the resonant frequency f2 of the receiver 604b. Similarly, the bandwidth 802b of the receiver 604b is narrow enough that it does not overlap with the resonant frequency f1 of the receiver 604a. Because of these characteristics, the receiver 604a with is narrow bandwidth 802a and frequency f1 does not respond to the frequency f2, and vice versa.

In some implementations, the receiver 604a having the bandwidth 802a may comprise a different shunt or series configuration (though not shown in this figure) than the receiver 604b. As a results, the receiver 604a may have a different Q factor than the receiver 604b. Accordingly, the bandwidth 802a of the receiver 604a may be longer or shorter (e.g., narrower or broader) than the bandwidth 802b of the receiver 604b, due at least in part to the shut or series configurations of the receivers 604a and 604b. In some embodiments, the quantity and/or quality of components forming the matching circuits of the receivers 604a and 604b may determine, at least in part, the Q factors, and, thus the bandwidths, of the receivers 604a and 604b.

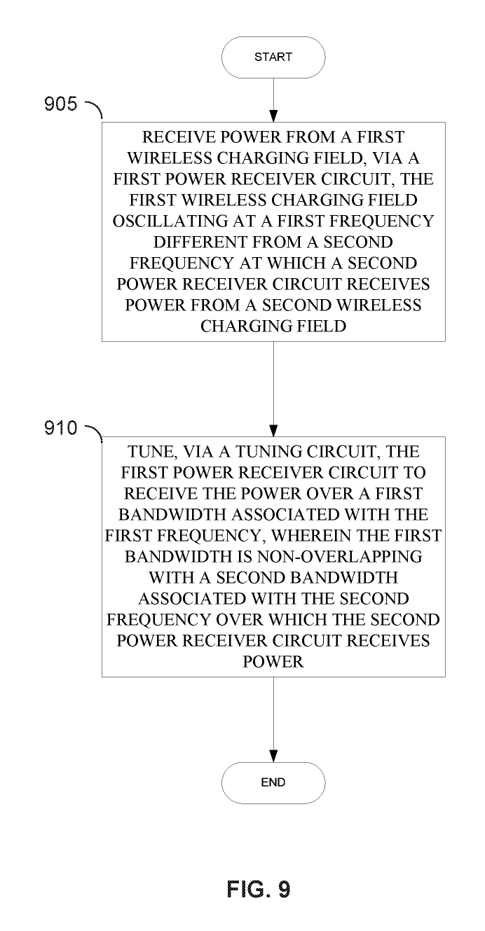

FIG. 9 is a process flow diagram of an exemplary method 900 for receiving wireless power, in accordance with certain aspects of the present disclosure. For example, the method could be performed by the receiver 500 illustrated in FIG. 5. Method 900 may also be performed by the implant 602a or 602b (FIG. 6) in some aspects. A person having ordinary skill in the art will appreciate that the method 900 may be implemented by other suitable devices and systems. Although the method 900 is described herein with reference to a particular order, in various aspects, blocks herein may be performed in a different order, or omitted, and additional blocks may be added.

Operation block 905 includes receiving power from a first wireless charging field, via a first power receiver circuit, the first wireless charging field oscillating at a first frequency different from a second frequency at which a second power receiver circuit receives power from a second wireless charging field. Operation block 910 includes tuning, via a tuning circuit, the first power receiver circuit to receive the power over a first bandwidth associated with the first frequency, wherein the first bandwidth is non-overlapping with a second bandwidth associated with the second frequency over which the second power receiver circuit receives power.

An apparatus for receiving wireless power may perform one or more of the functions of method 900, in accordance with certain aspects described herein. The apparatus may comprise a means receiving power. In certain aspects, the means for receiving power can be implemented by the receive antenna 504 (FIG. 5) or the receive antenna 218, or a transceiver. In certain aspects, the means for receiving power can be configured to perform the functions of block 905 (FIG. 9). The apparatus may comprise means for tuning the means for receiving power. In certain aspects, the means for tuning the means for receiving power can be implemented by the matching circuit 512 or a tuning circuit. In certain aspects, the means for tuning the means for receiving power can be configured to perform the functions of block 910 (FIG. 9).

The various operations of methods described above may be performed by any suitable means capable of performing the operations, such as various hardware and/or software component(s), circuits, and/or module(s). Generally, any operations illustrated in the Figures may be performed by corresponding functional means capable of performing the operations.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logical blocks, modules, circuits, and method steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the implementations.

The various illustrative blocks, modules, and circuits described in connection with the implementations disclosed herein may be implemented or performed with a general purpose hardware processor, a Digital Signal Processor (DSP), an Application Specified Integrated Circuit (ASIC), a Field Programmable Gate Array (FPGA) or other programmable logic device, discrete gate or transistor logic, discrete hardware components, or any combination thereof designed to perform the functions described herein. A general purpose hardware processor may be a microprocessor, but in the alternative, the hardware processor may be any conventional processor, controller, microcontroller, or state machine. A hardware processor may also be implemented as a combination of computing devices, e.g., a combination of a DSP and a microprocessor, a plurality of microprocessors, one or more microprocessors in conjunction with a DSP core, or any other such configuration.

The steps of a method and functions described in connection with the implementations disclosed herein may be embodied directly in hardware, in a software module executed by a hardware processor, or in a combination of the two. If implemented in software, the functions may be stored on or transmitted as one or more instructions or code on a tangible, non-transitory computer readable medium. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art. A storage medium is coupled to the hardware processor such that the hardware processor can read information from, and write information to, the storage medium. In the alternative, the storage medium may be integral to the hardware processor. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer readable media. The hardware processor and the storage medium may reside in an ASIC.

For purposes of summarizing the disclosure, certain aspects, advantages and novel features s have been described herein. It is to be understood that not necessarily all such advantages may be achieved in accordance with any particular implementation. Thus, the invention may be embodied or carried out in a manner that achieves or optimizes one advantage or group of advantages as taught herein without necessarily achieving other advantages as may be taught or suggested herein.

Various modifications of the above-described implementations will be readily apparent, and the generic principles defined herein may be applied to other implementations without departing from the spirit or scope of the application. Thus, the present application is not intended to be limited to the implementations shown herein but is to be accorded the widest scope consistent with the principles and novel features disclosed herein.

What is claimed is:

1. An apparatus for receiving wireless power, comprising:
a first power receiver circuit configured to receive power via coupling to a first wireless charging field oscillating at a first frequency different from a second frequency at which a second power receiver circuit receives power from a second wireless charging field; and
a tuning circuit coupled to and configured to tune the first power receiver circuit to receive the power over a first bandwidth associated with the first frequency, wherein the first bandwidth is non-overlapping with a second bandwidth associated with the second frequency over which the second power receiver circuit receives power.

2. The apparatus of claim 1, further comprising an implant device coupled to and configured to receive power from the first power receiver circuit, wherein the first power receiver circuit, the tuning circuit, and the implant device are implanted within an area of a body that is exposed to the first wireless charging field.

3. The apparatus of claim 2, wherein the first power receiver circuit is implanted within a bodily fluid or tissue in the body and wherein the first frequency is selected based on a type of bodily fluid or tissue in which the first power receiver circuit is implanted.

4. The apparatus of claim 2, wherein the first frequency of the first power receiver circuit is higher than the second frequency of the second power receiver circuit when the bodily fluid or tissue in which the first power receiver circuit is implanted has a higher optimal frequency than another bodily fluid or tissue in which the second power receiver circuit is implanted.

5. The apparatus of claim 1, wherein the first power receiver circuit is implanted within one of muscle tissue, bone tissue, organ tissue, blood tissue, or fat tissue in the body.

6. The apparatus of claim 1, wherein the first bandwidth is based on a quantity of power receiver circuits that receive power within an area of the first wireless charging field or based on a quantity of frequencies at which wireless charging fields are generated that also overlap an area of a body in which the first power receiver circuit is implanted.

7. The apparatus of claim 6, wherein the tuning circuit comprises one or more resonant capacitors in either a shunt or a series configuration and wherein the tuning circuit is configured to adjust the first bandwidth based on the quantity of power receiver circuits and a spectrum of frequencies.

8. The apparatus of claim 1, wherein the first frequency and the second frequency fall within a spectrum that ranges between 1.0 GHz and 2.4 GHz.

9. The apparatus of claim 1, wherein wireless field from which the first power receiver circuit receives power is a focused magnetic field within a near field or midfield region of a power transmitter.

10. The apparatus of claim 1, wherein the oscillating wireless charging field comprises a magnetic field that oscillates sinusoidally.

11. The apparatus of claim 1, wherein the first bandwidth is sufficiently narrow to avoid damage by a transmitter operating at the second frequency.

12. The apparatus of claim 1, wherein the first and second wireless charging fields are generated by one of a common transmitter or a first transmitter and a second transmitter, respectively.

13. A method of receiving power wirelessly, comprising:
receiving power from a first wireless charging field, via a first power receiver circuit, the first wireless charging field oscillating at a first frequency different from a second frequency at which a second power receiver circuit receives power from a second wireless charging field; and
tuning, via a tuning circuit, the first power receiver circuit to receive the power over a first bandwidth associated with the first frequency, wherein the first bandwidth is non-overlapping with a second bandwidth associated with the second frequency over which the second power receiver circuit receives power.

14. The method of claim 13, further comprising receiving power, via an implant device, from the first power receiver circuit, wherein the first power receiver circuit, the tuning circuit, and the implant device are implanted within an area of a body that is exposed to the first wireless charging field.

15. The method of claim 14, wherein the first power receiver circuit is implanted within a bodily fluid or tissue in the body and wherein the first frequency is selected based on a type of bodily fluid or tissue in which the first power receiver circuit is implanted.

16. The method of claim 14, wherein the first frequency of the first power receiver circuit is higher than the second frequency of the second power receiver circuit when the bodily fluid or tissue in which the first power receiver circuit is implanted has a higher optimal frequency than another bodily fluid or tissue in which the second power receiver circuit is implanted.

17. The method of claim 13, wherein the first power receiver circuit is implanted in one of muscle tissue, bone tissue, organ tissue, blood tissue, or fat tissue.

18. The method of claim 13, wherein the first bandwidth is based on a quantity of power receiver circuits that receive power within an area of the first wireless charging field or based on a quantity of frequencies at which wireless charging fields are generated that also overlap an area of a body in which the first power receiver circuit is implanted.

19. The method of claim 18, wherein the tuning circuit comprises one or more resonant capacitors in either a shunt or a series configuration, the method further comprising adjusting, via the tuning circuit, the first bandwidth based on the quantity of power receiver circuits and a spectrum of frequencies.

20. The method of claim 13, wherein the first frequency and the second frequency fall within a spectrum that ranges between 1.0 GHz and 2.4 GHz.

21. The method of claim 13, wherein the first power receiver circuit is further configured to receive power from a focused wireless field within a near field or midfield region of a power transmitter.

22. The method of claim 13, wherein the oscillating wireless charging field comprises a magnetic field that oscillates sinusoidally.

23. The method of claim 13, wherein the first bandwidth is sufficiently narrow to avoid damage by a transmitter operating at the second frequency.

24. The method of claim 13, wherein the first and second wireless charging fields are generated by one of a common transmitter or a first transmitter and a second transmitter, respectively.

25. An apparatus for receiving wireless power, comprising:
means for receiving power from a first wireless charging field, the first wireless charging field oscillating at a first frequency different from a second frequency at which a power receiver receives power from a second wireless charging field; and
means for tuning the means for receiving power to receive the power over a first bandwidth associated with the first frequency, wherein the first bandwidth is non-overlapping with a second bandwidth associated with the second frequency over which the power receiver receives power.

26. The apparatus of claim 25, further comprising means for implanting the means for receiving power and the means for tuning within an area of a body that is exposed to the first wireless charging field.

27. The apparatus of claim 26, wherein the means for implanting is implanted within a bodily fluid or tissue in the body and wherein the first frequency is selected based on a type of bodily fluid or tissue in which the means for implanting is implanted.

28. A system for transferring wireless power, the system comprising:
a first power receiver circuit configured to receive a first power via coupling to a first wireless charging field oscillating at a first frequency, the first power receiver circuit comprising a first tuning circuit configured to tune the first power receiver circuit to receive the first power over a first bandwidth associated with the first frequency; and
a second power receiver circuit configured to receive a second power via coupling to a second wireless charging field oscillating at a second frequency different from the first frequency, the second power receiver circuit comprising a second tuning circuit configured to tune the second power receiver circuit to receive the second power over a second bandwidth associated with the second frequency, wherein the second bandwidth is non-overlapping with the first bandwidth associated with the first frequency over which the first power receiver circuit receives power.

29. The system of claim 28, further comprising at least one of a common power transmitter configured to generate both the first wireless charging field at the first frequency and the second wireless charging field at the second frequency or a first power transmitter configured to generate the first wireless charging field at the first frequency and generate the second wireless charging field at the second frequency.

30. The system of claim 29, wherein the first and second tuning circuits are configured to tune the first and second power receiver circuits, respectively, to avoid damage of the first and second power receiver circuits from the respective wireless charging field of the other tuning circuit.
